(19) United States
(12) Patent Application Publication
(10) Pub. No.: US 2005/0144738 A1
(43) Pub. Date: Jul. 7, 2005

(54) Method for double color dyeing

(76) Inventor: Shin-Chang Wu, Taoyuan City (TW)

Correspondence Address: Shin-Chang Wu, P.O. Box No. 6-57, Junghe, Taipei 235, TW

(21) Appl. No.: 10/745,672

(22) Filed: Dec. 29, 2003

(51) Int. Cl. A61K 7/13

(52) U.S. Cl. 8/405

(57) ABSTRACT

The present invention embodies a dyeing method applied to textiles or yarn, and primarily encompasses imprinting prearranged patterns of an ionization agent onto the textile or the yarn, and thereafter drying the ionization agent and thereby setting a pattern design thereon. Afterward, during a dyeing process of the textile or yarn, altering characteristics of ion composition through the ionization agent or utilizing chemical reaction effect of dyestuffs having differing properties intensifies color of dyestuffs adhering to pattern areas formed, and thereby enables a finished dyed product to acquire a pronounced double color display effect of ionization agent areas and dye areas.

### BACKGROUND OF THE INVENTION

(a) Field of the Invention

The present invention relates to a dyeing method for textiles or yarn, and more particularly to realizing production of a finished product that acquires a pronounced double color display effect of ionization agent areas and dye areas.

(b) Description of the Prior Art

Present applied dyeing art of textile fabrics constitutes firstly weaving yarn into cloth, and thereafter dyeing the woven cloth by employing dip dyeing or overstain methods. Another process comprises firstly employing the dip dyeing method to dye the yarn prior to weaving the cloth, and thereafter apply weaving techniques to form a color combination in the cloth.

However, no matter whether the product being dyed is cloth already woven or yarn material for use prior to weaving, during entire process of overstain or dip dyeing, because of adherence of dyestuffs, all areas that come into contact with the dyestuffs are dyed thereat. Therefore, conventional mass automation dyeing art is only able to achieve a finished product displaying a single color, which is then used as material for follow-up weaving.

Consequences of above are such that when the textile fabric needs providing with a hue of differing colors and a pattern display effect, completion of such requires necessity for a weaving machine of some complexity, and thereby increases manufacturing cost of whole process. Moreover, such a convoluted processing procedures reduces productivity of the textile fabrics, and produces an overage of rejects.

Nevertheless, utilizing dyestuffs to imprint other patterns having a hue display effect directly on a surface of the textile fabric does achieve an increased hue display effect with a lower manufacturing cost. However, the textile fabric manufactured by such a method of imprinting not only possess poor qualitative feeling, but also the dyestuffs easily peel off during course of washing and usage of the imprinted textile fabric, thus spoiling integral pattern of the original textile fabric, and seriously affecting beauty of the textile fabric itself.

### SUMMARY OF THE INVENTION

The present invention embodies imprinting prearranged patterns of an ionization agent onto a textile, and thereafter drying the ionization agent, and thereby setting a pattern design thereon. Afterward, during a dyeing process of the textile, altering characteristics of ion composition through the ionization agent or utilizing chemical reaction effect of dyestuffs having differing properties intensifies color of dyestuffs adhering to pattern areas formed, and thereby enables a finished dyed product to acquire a pronounced double color display effect of ionization agent areas and dye areas.

Furthermore, the ionization agent can also be imprinted onto smoothed out yarn, which then undergoes an equivalent drying and dyeing process, and thereby enables the yarn to achieve the pronounced double color display effect of the ionization agent areas and the dye areas. And the finished textile woven from such yarn thereby acquires a double color distribution effect directly from the ionization agent areas and the dye areas of the yarn formed therein.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view of a first embodiment of a textile imprinted with an ionization agent according to the present invention.

FIG. 2 shows a schematic view of the first embodiment of a textile after dyeing according to the present invention.

FIG. 3 shows a schematic view of a second embodiment of yarn after dyeing according to the present invention.

FIG. 4 shows a schematic view of the second embodiment of the yarn woven into the textile according to the present invention.

FIG. 5 shows a schematic view of the second embodiment of the yarn compete and pattern finalized with the imprinted and dried ionization agent according to the present invention.

FIG. 6 shows a schematic view of the second embodiment of the yarn furled onto a tiled wheel according to the present invention.

### DETAILED DESCRIPTION OF THE PREFERRED EMBODIMENTS

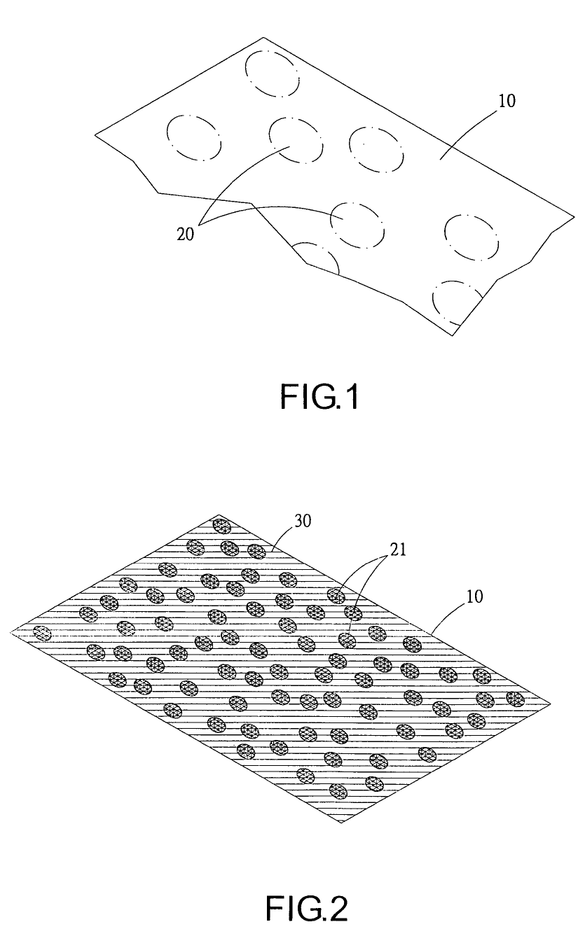

Referring to FIGS. 1 and 2, which show a method for double color dyeing of the present invention having an underlying technique comprising imprinting of prearranged patterns of a hydrophilic ionization agent 20 onto a textile 10. In an embodiment as depicted in FIG. 1, the ionization agent 20 is of a nature that is transparent and colorless, therefore imaginary lines indicate pattern areas formed by the ionization agent 20.

Following aforementioned, the finished textile 10 having patterns imprinted thereon is heated at a temperature between 60° C.-90° C., thereby drying the ionization agent 20 on the textile and setting a pattern design thereon. Thereafter, a dyeing process of the textile 10 is carried out. During the dyeing process of the textile 10, altering characteristics of ion composition through the ionization agent 20 or utilizing chemical reaction effect of dyestuffs having differing properties (such as acid properties of acid dyes) intensifies color of dyestuffs adhering to the pattern areas formed, and thereby enables a finished product of the dyed textile 10 (see FIG. 2) to acquire a pronounced double color display effect of the ionization agent areas 21 and dye areas 30.

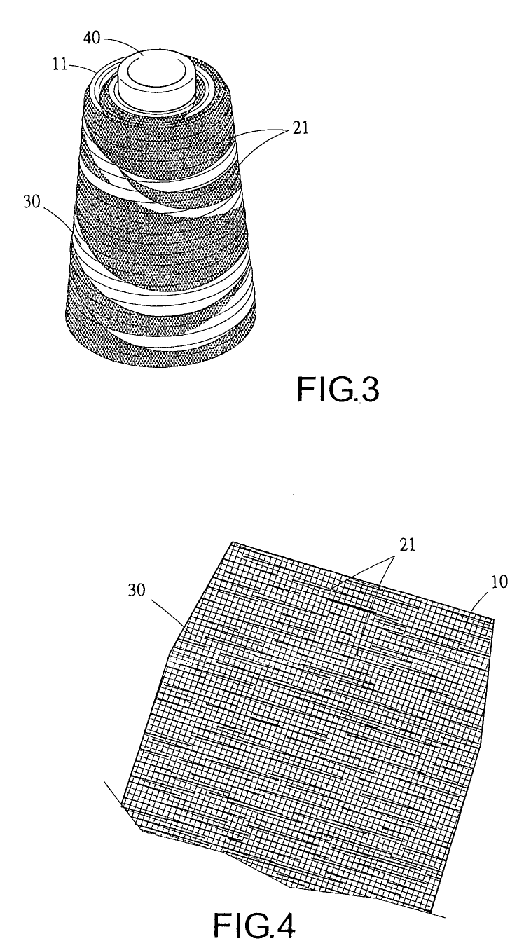

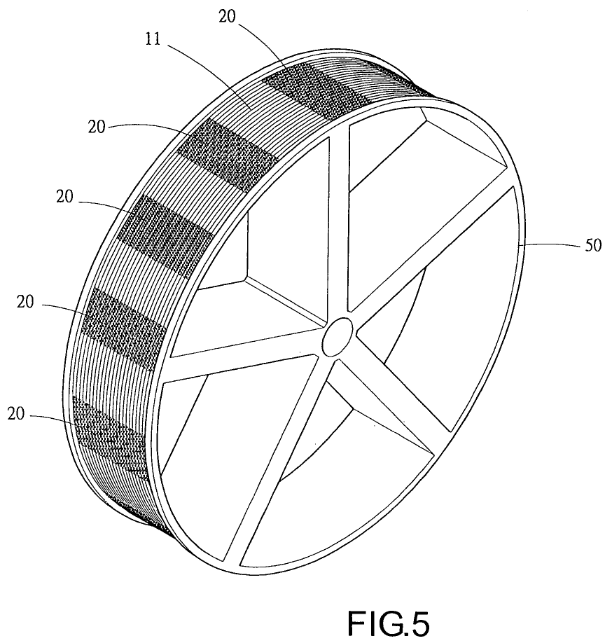

Furthermore, referring to FIG. 3 and 4, which show that implementation of the method for double color dyeing of the present invention can also be applied to the dyeing process of yarn, thereby allowing the yarn 11 to acquire the pronounced double color display effect of the ionization agent areas 21 and the dye areas 30, and enabling the textile 10 so woven from the yarn 11 to achieve double color distribution effect directly from the ionization agent areas 21 and the dye areas 30 of the yarn 11. Wherein, as depicted in FIG. 5, prior to dyeing the yarn 11, the ionization agent 20 is imprinted onto the smoothed out yarn 11, which then undergoes an equivalent drying procedure. As depicted in FIG. 5, the pattern areas formed from the ionization agent 20, after undergoing drying to set the pattern design, traces of the ionization agent 20 on the yarn 11 are intensified thereof. Whereupon the completely dry yarn 11 is wound onto a yarn tube 40 (see FIG. 3), and thereafter the dyeing process is carried out. The yarn 11 thereby acquires a pronounced double color display effect of the ionization areas 21 and the dye areas 30.

Moreover, conventional knitting machines or other spinning and weaving machines when proceeding with spinning, weaving, embroidering, and so on, are enabled for continuous drawing-out of the yarn during course of operations. Whereupon, majority of yarn manufacturers wind the yarn 11 onto a cardboard yarn tube 40, as depicted in FIG. 3, which thereby facilitates mounting the yarn tube 40 onto the knitting machine or other spinning and weaving machinery, and usage thereof. Therefore, the present invention utilizes smoothing out and winding of the yarn 11 directly onto a rim of a tiling wheel 50 during production of the yarn 11, as depicted in FIG. 5, and thereby facilitates proceeding with follow-up operations including imprinting and drying of the ionization agent 20. Lastly, the completely dry yarn 11 is wound onto the yarn tube 40, and the dyeing process is proceeded with thereafter.

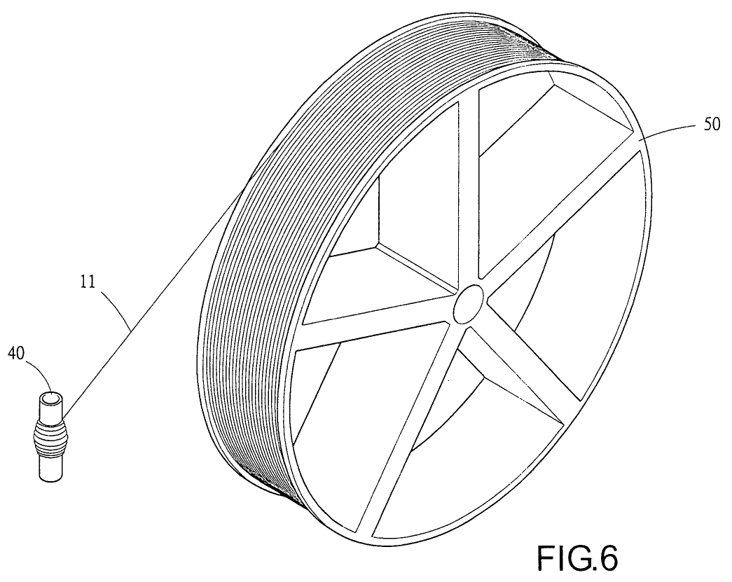

In addition, the present invention can also be employed to implement dyeing of yarn sold retail or supplied through the yarn manufacturers. Referring to FIG. 6, the yarn 11 of the yarn tube 40 is firstly smoothed out and wound onto the rim of the tiling wheel 50, and after processing steps including the imprinting and drying of the ionization agent 20, the dried yarn 11 is wound onto the yarn tube 40, and the follow-up dyeing process is proceeded with thereafter.

In conclusion, as disclosed above, the method for double color dyeing of the present invention actualizes providing the textile 10 or the yarn 11 with the double color display effect of the pronounced ionization agent areas 21 and the dye areas 30. It is of course to be understood that the embodiments described herein is merely illustrative of the principles of the invention and that a wide variety of modifications thereto may be effected by persons skilled in the art without departing from the spirit and scope of the invention as set forth in the following claims.

1. A method for double color dyeing, comprising imprinting of prearranged patterns of an ionization agent onto yarn, and thereafter drying the ionization agent and thereby setting a pattern design thereon; during a dyeing process of the yarn, altering characteristics of ion composition through the ionization agent or utilizing chemical reaction effect of dyestuffs having differing properties intensifies color of dyestuffs adhering to pattern areas formed, and thereby enables a finished dyed product to acquire a pronounced double color display effect of the ionization agent areas and dye areas.

2. A method for double color dyeing, comprising imprinting of prearranged patterns of the ionization agent onto the textile, and thereafter drying the ionization agent and thereby setting a pattern design thereon; during the dyeing process of the textile, altering characteristics of ion composition through the ionization agent or utilizing chemical reaction effect of dyestuffs having differing properties intensifies color of dyestuffs adhering to pattern areas formed, and thereby enables the finished dyed product to acquire a pronounced double color display effect of the ionization agent areas and the dye areas.

3. The method for double color dyeing according to claim 1, wherein the ionization agent is hydrophilic.

4. The method for double color dyeing according to claim 2, wherein the ionization agent is hydrophilic.

5. The method for double color dyeing according to claim 1, wherein the ionization agent is dried at a temperature between 60° C.-90° C.

6. The method for double color dyeing according to claim 2, wherein the ionization agent is dried at a temperature between 60° C.-90° C.

7. The method for double color dyeing according to claim 1, wherein a smoothing out method is employed to wind the yarn directly onto a rim of a tiling wheel, and processing of pattern imprinting and drying of the ionization agent is proceeded with thereafter.

8. The method for double color dyeing according to claim 1, wherein the completely dry yarn s wound onto a yarn tube, and the dyeing process is proceeded with thereafter.
